(19) United States
(12) Patent Application Publication
(10) Pub. No.: US 2004/0105875 A1
(43) Pub. Date: Jun. 3, 2004

(54) Composition containing a secondary or tertiary carbonyl amine, method of use thereof, compounds

(75) Inventors: Christophe Boulle, Lagny S/Marine (FR); Maria Dalko, Gif S/Yvette (FR); Alexandre Cavezza, Tremblay-En-France (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/670,327

(22) Filed: Sep. 26, 2003

Related U.S. Application Data
(60) Provisional application No. 60/426,375, filed on Nov. 15, 2002.

(30) Foreign Application Priority Data
Oct. 3, 2002 (FR) 0212261

(51) Int. Cl. A61K 7/00; A61K 31/17

(52) U.S. Cl. 424/401; 514/649; 514/588

(57) ABSTRACT

The present invention relates to a cosmetic process for treating wrinkled skin, by topical application to the skin of a composition containing, in a physiologically acceptable medium, at least one secondary or tertiary carbonyl amine of formula (I):

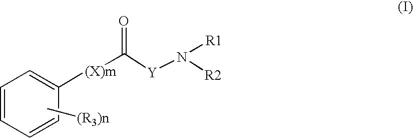

The invention also relates to a novel family of carbonyl amines, and also to compositions containing them.

### REFERENCE TO PRIOR APPLICATIONS

This application claims priority to U.S. provisional application 60/426,375 filed Nov. 15, 2002, and to French patent application 0212261 filed Oct. 3, 2002, both incorporated herein by reference.

### FIELD OF THE INVENTION

The present invention relates in particular to a process for treating wrinkled skin, comprising the topical application to the skin of a composition, which is suitable for topical application to the skin, comprising, in a physiologically acceptable medium, at least one secondary or tertiary carbonyl amine of formula (I):

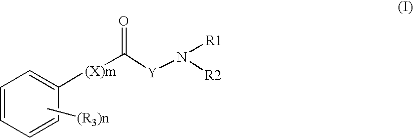

where variables n, m, R, X and Y are defined herein.

The invention also relates to a novel family of carbonyl amines, and also to compositions, preferably cosmetic compositions, containing them.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women, and even men, currently have a tendency to wish to look youthful for as long as possible and consequently seek to eradicate the signs of ageing of the skin, which are reflected especially by wrinkles and fine lines. In this respect, the media and the fashion world report about products intended to maintain for as long as possible skin which is radiant and wrinkle-free, which are signs of youthful skin, and all the more so since the physical appearance acts on the psyche and/on the morale.

Hitherto, wrinkles and fine lines were treated with cosmetic products containing active agents acting on the skin, for example by moisturizing it or improving its cell renewal or alternatively by promoting the synthesis or preventing the degradation of the elastic fibres of which skin tissue is composed.

Although these treatments make it possible to act on the wrinkles and fine lines caused by chronological or intrinsic ageing, and also on those caused by photo-ageing, they have no effect on the expression wrinkles and fine lines, which require an intervention on the muscular contractile component of the wrinkles present in the skin.

Hitherto, the only means commonly used for acting on expression wrinkles is botulinum toxin, which is especially injected into the wrinkles of the glabella, which are the wrinkles between the eyebrows (see J. D. Carruters et al., J. Dermatol. Surg. Oncol., 1992, 18, pp. 17-21).

The Assignee has also proposed various compounds capable of affording a dermo-decontracting effect when they are applied topically to the skin, thus making is possible to act on expression wrinkles via another route. Among these compounds, mention may be made especially of antagonists of the receptors associated with the calcium channels (FR-2 793 681), and in particular manganese and its salts (FR-2 809 005) and alverine (FR-2 798 590); and agonists of the receptors associated with the chlorine channels, including glycine (EP-0 704 210) and certain extracts of Iris pallida (FR-2 746 641).

However; there is still a need for compounds that are effective in smoothing out or eliminating expression wrinkles and fine lines.

### DETAILED DESCRIPTION OF THE PREFERRED EMBODIMENTS

The inventors have now discovered, surprisingly, that certain secondary and tertiary amines can satisfy this need.

Admittedly, document EP-1 090 630 discloses certain secondary and tertiary amines having the property of increasing collagen synthesis by the fibroblasts and of moisturizing the skin, which are useful against dry skin and atopic dermatitis, and which also show efficacy on wrinkles. However, the carbonyl amines cited in the said document do not comprise a phenyl group and are such that the carbonyl group is directly adjacent to the nitrogen atom. In addition, they have no effect on expression wrinkles and fine lines.

Moreover, document WO 93/05763 discloses certain amines that are di- and trisubstituted with at least two chains each bearing at least one hydroxyl group. These amines increase the differentiation of keratinocytes, limit the UV-induced thickening of the epidermis and are useful for preventing and treating wrinkles induced by UVB radiation. It is not suggested that these amines, which are different from those that are the subject of the present invention in the sense that they do not comprise a carbonyl group, have any effect on expression wrinkles and fine lines.

Similarly, document EP-0 691 327 discloses a very broad family of mono-, di- or trisubstituted amines that are described as being effective for smoothing out wrinkles. The amines given as examples in the said patent application are not substituted with chains capable of comprising a carbonyl group, in contrast with the amines that are the subject of the present invention. In addition, it is not suggested that they have any effect on expression wrinkles and fine lines.

Still other carbonyl amines have been described in document U.S. Pat. No. 6,372,772. These compounds differ from those that are the subject of the present invention in the sense that the chain linking the carbonyl group to the nitrogen atom comprises a methylene substituent. This methylene group, conjugated with the carbonyl group and positioned as a substituent of the chain, constitutes a good Michaël receptor, which is capable of scavenging radicals and nucleophiles in general. It gives the molecule photo-reactivity that is not desirable for cosmetic applications, insofar as it may cause problems of harmfulness.

The inventors have now discovered that certain secondary and tertiary carbonyl amines make it possible to obtain compositions that are effective for smoothing out expression wrinkles and fine lines.

Some of these amines have already been described, for example in the publication by Halise Inci Gul et al., Antifungal Evaluation of bis Mannich Bases derived from Acetophenones and their Corresponding Piperidinols and Stability Studies, Biological and Pharmaceutical Bulletin, Vol. 25, No. 10, pp. 1307-1310 (2002), for instance antifungal agents that may be used against dermatophytes.

Other amines included in the general structure of the amines according to the present invention have been described as muscle relaxants by acting on the central nervous system (WO 95/18092 and Chawla H. P. S. et al., Agents acting on the Central Nervous System, XII. 3-Tert-Aminopropiophenones as central muscle relaxants and diuretics”, Journal of Medicinal Chemistry, 13(3), 480-8 (1970)). However, it was not foreseeable that these compounds would have a relaxing action on fibroblasts, making it possible to envisage their topical use on the skin to reduce expression wrinkles.

Admittedly the Assignee has previously described the use of alverine, which is a trisubstituted amine, as a dermo-decontracting agent intended to smooth out expression wrinkles. However, in contrast with the compounds that are the subject of the present invention, alverine does not contain a carbonyl group. Now, it was not obvious that the dermo-decontracting activity of alverine would be conserved and even improved by introducing carbonyl groups into its molecule.

One subject of the present invention is thus a process for treating wrinkled skin, preferably a cosmetic process, comprising the topical application to said skin of a composition, which is preferably suitable for topical application to the skin, comprising, in a physiologically acceptable medium, at least one compound of formula (I):

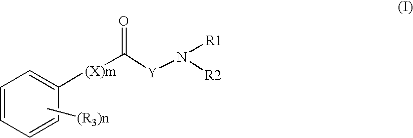

in which:

R₁ denotes a hydrogen atom or a linear or branched, saturated or unsaturated C₁-C₈ alkyl group,

R₂ denotes:

a linear, branched or cyclic, saturated or unsaturated C₁-C₂₀ alkyl group, optionally substituted with an ═O group, and/or with one or more —CN, —OR, —SR, —NRR′, —COR, —COOR, —CONRR′, —NR—CO—R′, —NR—CO—NR′R″ or —CF₃ groups and/or with one or more halogen atoms and/or with a phenyl group optionally substituted with a C₁-C₄ alkyl group, a C₁-C₄ alkoxy group, a hydroxyl group, a halogen atom and/or a CF₃ group,

an aryl group optionally substituted with at least one of a C₁-C₄ alkyl group, a C₁-C₄ alkoxy group, a hydroxyl group, a halogen atom or a CF₃ group, or

a heterocycle,

in which R, R′ and R″ independently denote a hydrogen atom or a linear or branched C₁-C₄ alkyl group, or an aryl group,

R₃ denotes a linear, branched or cyclic, saturated or unsaturated C₁-C₁₂ alkyl group, or an aryl group, optionally substituted with a hydroxyl group, a C₁-C₄ alkoxy group or a hydrogen atom, a —CN, —OR, —SR, —NRR′, —COR, —COOR, —CONRR′, —NR—CO—R′, —NR—CO—NR′R″ or —CF₃ group or a halogen atom,

in which R, R′ and R″ have the meanings given above,

—X is a saturated or unsaturated, optionally substituted linear C₁-C₉ alkylene group,

Y is a saturated or unsaturated, optionally substituted linear C₁-C₁₀ alkylene group,

the optional one or plural substituents of X and Y being chosen independently from —OR, —SR, —NRR′, —COR, —COOR, —CO—NRR′, —NR—CO—R′R″, CF₃ groups, and mixtures thereof,

in which R, R′ and R″ have the meanings given above,

m is 0 or 1,

n is 0, 1, 2, or 3,

or the addition salt thereof with an acid.

In formula (I), the alkyl groups may preferably be chosen, depending on the case, from the following groups: methyl, ethyl, n-propyl, isopropyl, n-butyl, isobutyl, tert-butyl, pentyl, hexyl, heptyl, octyl, nonyl, decyl, undecyl, dodecyl, myristyl, palmityl, stearyl and arachidyl.

For its part, the aryl group is preferably a phenyl group.

The alkylene groups may preferably be methylene, ethylene, propylene, butylene, pentylene, hexylene, heptylene, octylene, nonylene or decylene groups.

The halogen atom may preferably be a fluorine, chlorine, bromine or iodine atom.

The heterocycle may preferably be a saturated or unsaturated heterocyclic ring containing from one to three heteroatoms selected from oxygen, nitrogen and sulphur.

Salts of the compound of formula (I) that are preferred include the salts obtained by addition of the compound of formula (I) with a mineral acid, chosen preferably from hydrochloric acid, sulphuric acid, nitric acid and phosphoric acid, or with an organic acid, chosen in particular from succinic acid, fumaric acid, lactic acid, glycolic acid, citric acid and tartaric acid.

The compounds of formula (I) may be prepared as described in Badosov. E. P. et al., Chemistry of β-Amino Ketones, VII. Synthesis of Substituted Methyl and Phenyl β-[N-methyl-N(β-acetylethyl)]aminoethyl ketones by aminomethylation of ketones with formaldehyde and the salts of methyl and phenyl β-methylaminoalkyl ketones, Zhurnal Organicheskoi Khimii, Vol. 11, No. 5, pp. 972-977, May 1975. The synthesis of these compounds has moreover been described in Von K. Thiele et al., Neue Piperidinderivative aus herzwirksamen—Aminoketonen, Arzneim. Forsch., 18(10), 1263-9 (1968). Such synthesis is within the skill of the ordinary artisan in view of this disclosure.

According to one preferred embodiment of the invention, the compound of formula (I) is such that at least one of the following conditions, and preferably two or more, and more preferably all these conditions, is(are) satisfied:

m=0

n=0

Y is a linear C₁-C₃ alkylene group,

R₁ is a C₁-C₃ alkyl group,

R₂ is a C₁-C₃ alkyl group substituted with an arylcarbonyl group in formula (I), or the salt thereof with a mineral acid.

Even more preferably, the compound of formula (I) is such that:

m=0

n=0

Y is an ethylene group,

R₁ is a methyl group, and

R₂ is an ethyl group substituted with a benzoyl group

in formula (I), or the salt thereof with hydrochloric acid.

Such a compound, which corresponds to the formula (Ia) below:

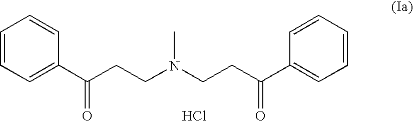

is available especially from the company Salor under the commercial reference S35,861-4. As a variant, it may be prepared by aminomethylation of acetophenone using 3-methylamino-1-phenyl-1-propanone hydrochloride (which is itself obtained by reacting methylamine with phenyl isopropenyl ketone) and formaldehyde, as described in the first publication indicated above.

Other examples of compounds of formula (I) that are useful in the present invention comprise compounds (Ib) to (Ii) below:

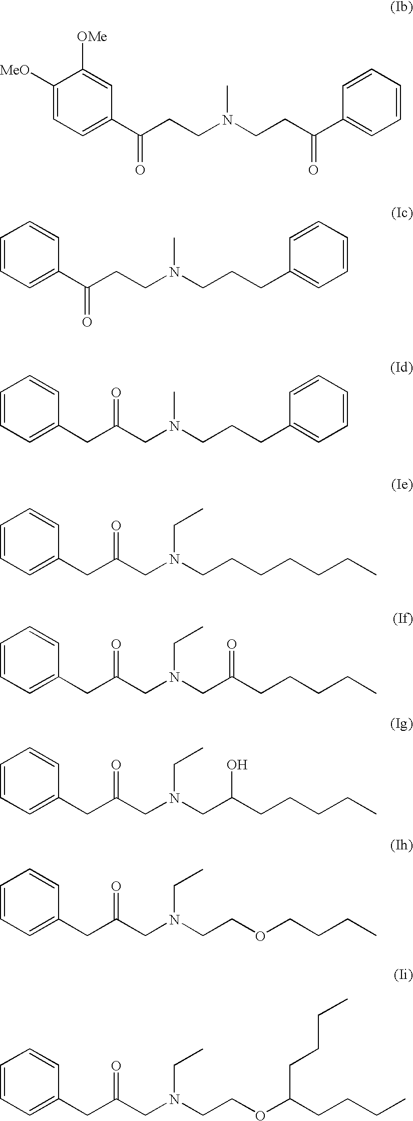

As will be demonstrated in the Examples below, the inventors have demonstrated a dermo-decontracting effect of the compounds of formula (I) according to the invention, which makes them useful in smoothing out expression wrinkles and fine lines.

A subject of the invention is thus also the use of at least one compound of formula (I) as defined above, in a composition that is suitable for topical application to the skin, as an agent for smoothing out wrinkles and fine lines, in particular expression wrinkles and fine lines.

The inventors have moreover demonstrated that some of the compounds of formula (I) are novel and show advantageous dermo-decontracting activity.

A subject of the invention is thus also a subfamily of compounds of formula (I), which correspond to the formula (II) below:

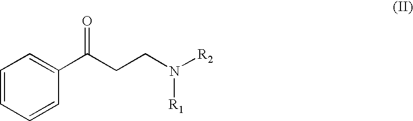

in which:

R₁ is a hydrogen atom or a methyl or ethyl group,

R₂ is a linear or branched C₆-C₂₀ alkyl radical which may be substituted with one or more of an oxo group and/or a hydroxyl group, and also the addition salts thereof with an acid.

The acid used to salify the compounds of formula (II) preferably is a physiologically acceptable acid, as defined above.

Examples of compounds of formula (II) correspond to formulae (IIa) to (IIe) below:

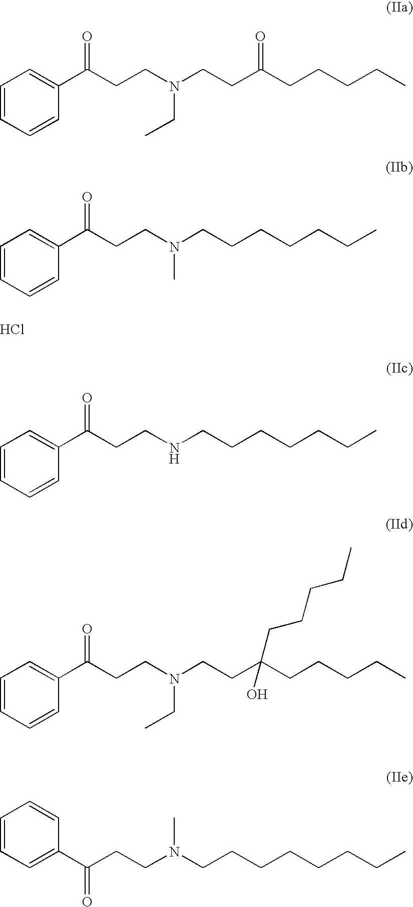

These compounds may be prepared according to the following reaction scheme:

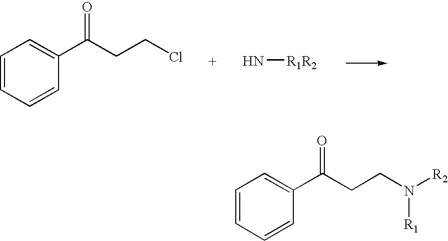

by nucleophilic substitution, in methanol at room temperature, with magnetic stirring. The product obtained may be worked up and purified on silica.

In the case of the compound of formula (IId), since the starting amine is not commercially available, the preparation process described in Example 1 below may be used.

A subject of the present invention is also a second subfamily of novel compounds, included in the general formula (I) and corresponding to formula (III) below:

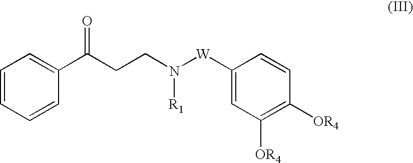

in which:

R₁ denotes a hydrogen atom or a methyl group,

W denotes a linear C₁-C₅ alkylene chain, which may be substituted with one or more of an oxo group and/or a hydroxyl group,

R₄ denotes a hydrogen atom or a methyl group, it being understood that the compound of formula (III) does not comprise an amide function,

and also the addition salts thereof with an acid.

The acid used to salify the compounds of formula (III) preferably is a physiologically acceptable acid, as defined above.

Examples of compounds of formula (III) correspond to formulae (IIIa) and (IIIb) below:

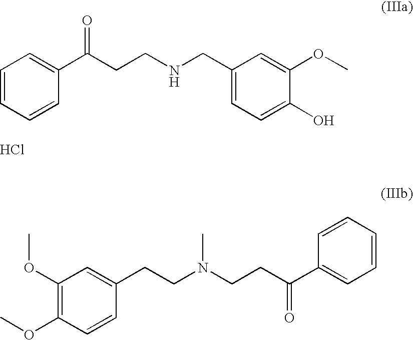

These compounds may be prepared according to a process analogous to the process for preparing the compounds of formula (II) above.

A subject of the present invention is also a composition, which is in particular suitable for topical application to the skin, comprising, in a physiologically acceptable medium, at least one compound corresponding to formula (II) or (III) above, in particular at least one of the compounds (IIa) to (IId), (IIIa) and (IIIb).

The amount of compound(s) of formula (I)—and thus of compounds of formulae (II) and (III)—which may be used according to the invention is not limited and depends on the desired effect, and may thus vary within a wide range.

To give a general order of magnitude, these compounds may be used in an amount representing from 0.01% to 10% of the total weight of the composition, and more, preferably in an amount representing from 0.05% to 5% of the total weight of the composition and more preferably in an amount representing from 0.1% to 2% of the total weight of the composition.

The composition according to the invention is preferably suitable for topical application to the skin and thus preferably contains a physiologically acceptable medium, i.e. a medium that is compatible with the skin and optionally with its integuments (eyelashes, nails and hair) and/or mucous membranes. This medium is advantageously cosmetically acceptable, i.e. it does not cause any itching, stinging or redness liable to put the user off the composition, and it has a pleasant appearance, odour and feel.

This composition may be in any presentation form, and preferably is in a form normally used in cosmetics It may especially be in the form of an optionally gelled aqueous solution, a dispersion of the lotion type, optionally a two-phase lotion, an emulsion obtained by dispersing a fatty phase in an aqueous phase (O/W emulsion) or conversely (W/o emulsion), or a triple emulsion (W/O/W or O/W/O emulsion) or a vesicular dispersion of ionic and/or nonionic type. These compositions are prepared according to the usual methods. A composition in the form of an oil-in-water emulsion is preferably used according to this invention.

The invention composition may be more or less fluid and may have the appearance of a white or coloured cream, an ointment, a milk, a lotion, a serum, a paste or a mousse. It may optionally be applied in the form of an aerosol. It may also be in solid form, in particular in the form of a stick. It may be used as a care product, and/or as a makeup product for the skin.

The composition according to the invention may also contain adjuvants such as those that are common in cosmetics, such as hydrophilic or lipophilic gelling agents, hydrophilic or lipophilic active agents, preserving agents, antioxidants, solvents, fragrances, fillers, screening agents, pigments, odour absorbers and dyestuffs. The amounts of these various adjuvants are those conventionally used in the field under consideration, and, for example, from 0.01% to 20% relative to the total weight of the composition. Depending on their nature, these adjuvants may be introduced into the fatty phase, into the aqueous phase, or into lipid vesicles. In any case, these adjuvants, and also the proportions thereof, should preferably be chosen so as not to harm the desired properties of the compounds of formula (II) or (III) according to the invention.

When the composition according to the invention is an emulsion, the proportion of the fatty phase may range from 5% to 80% by weight and preferably from 5% to 50% by weight relative to the total weight of the composition. The oils, emulsifiers and co-emulsifiers used in the composition in emulsion form are chosen from those conventionally used in the field under consideration. The emulsifier and co-emulsifier are preferably present in the composition in a proportion ranging from 0.3% to 30% by weight and more preferably from 0.5% to 20% by weight relative to the total weight of the composition.

As oils which may be used in the invention, included are mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax or ozokerite) may also be used as fatty substances.

As examples of emulsifiers and co-emulsifiers that may be used in the invention, included are fatty acid esters of polyethylene glycol such as PEG-100 stearate, and fatty acid esters of glycerol such as glyceryl stearate.

Hydrophilic gelling agents that may be mentioned in particular include carboxyvinyl polymers (carbomer), acrylic copolymers such as acrylate/alkylacrylate copolymers, polyacrylamides, polysaccharides, natural gums and clays, and lipophilic gelling agents that may be mentioned include modified clays, for instance bentones, metal salts of fatty acids, hydrophobic silica and polyethylenes.

As active agents, it will be advantageous to introduce into the composition according to the invention at least one compound chosen from: desquamating agents; moisturizers; depigmenting or propigmenting agents; antiglycation agents; NO-synthase inhibitors; agents for stimulating the synthesis of dermal or epidermal macromolecules and/or for preventing their degradation; agents for stimulating the proliferation of fibroblasts and/or keratinocytes or for stimulating the differentiation of keratinocytes; dermo-decontracting agents; tensioning agents; antipollution agents and/or free-radical scavengers; agents acting on the capillary circulation; agents acting on the energy metabolism of cells; and mixtures thereof.

Examples of such additional compounds are given below.

1. Desquamating Agents

The term “desquamating agent” means any compound capable of acting:

either directly on the desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;

or on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; amino-sulphonic compounds and in particular (N-2-hydroxyethylpiperazine-N-2-ethane)sulphonic acid (HEPES); 2-oxothiazolidine-4-carboxylic acid (procysteine) derivatives; α-amino acid derivatives of the type such as glycine (as described in EP-0 852 949, and sodium methyl glycine diacetate sold by BASF under the trade name Trilon M); honey; sugar derivatives such as O-octanoyl-6-D-maltose and N-acetylglucosamine.

2. Moisturizer

The term “moisturizer” means:

either a compound acting on the barrier function, in order to maintain the moisturization of the stratum corneum, or an occlusive compound. Mention may be made of ceramides, sphingoid-based compounds, lecithins, glycosphingolipids, phospholipids, cholesterol and its derivatives, phytosterols (stigmasterol, β-sitosterol or campesterol), essential fatty acids, 1,2-diacyl-glycerol, 4-chromanone, pentacyclic triterpenes such as ursolic acid, petroleum jelly and lanolin;

or a compound that directly increases the water content of the stratum corneum, such as threalose and its derivatives, hyaluronic acid and its-derivatives, glycerol, pentanediol, sodium pidolate, serine, xylitol, sodium lactate, polyglyceryl acrylate, ectoin and its derivatives, chitosan, oligosaccharides and polysaccharides, cyclic carbonates, N-lauroyl-pyrrolidonecarboxylic acid and N-α-benzoyl-L-arginine;

or a compound that activates the sebaceous glands, such as DHEA and its derivatives and vitamin D and its derivatives.

3. Depigmenting or Propigmenting Agent

The depigmenting agents that may be incorporated into the composition according to the present invention comprise, for example, the following compounds: kojic acid; ellagic acid; arbutin and its derivatives such as those described in patent applications EP-895 779 and EP-524 109; hydroquinone; aminophenol derivatives such as those described in patent applications WO 99/10318 and WO 99/32077, and in particular N-cholesteryloxycarbonyl-para-aminophenol and N-ethyloxycarbonyl-para-aminophenol; iminophenol derivatives, in particular those described in patent application WO 99/2207; L-2-oxothiazolidine-4-carboxylic acid or procysteine, and also its salts and esters; ascorbic acid and its derivatives, especially ascorbyl glucoside; and plant extracts, in particular extracts of liquorice, of mulberry and of skullcap, without this list being limiting.

Propigmenting agents that may be mentioned include the extract of burnet (Sanguisorba officinalis) sold by the company Maruzen, and extracts of chrysanthemum (Chrysanthemum morifolium).

4. Anti-Glycation Agent

The term “anti-glycation agent” means a compound for preventing and/or reducing the glycation of skin proteins, in particular of dermal proteins such as collagen.

Examples of anti-glycation agents are plant extracts of the Ericacea family, such as an extract of blueberry (Vaccinium angustifolium); ergothioneine and its derivatives; and hydroxystilbenes and their derivatives, such as resveratrol and 3,3′,5,5′-tetra-hydroxystilbene.

5. NO-Synthase Inhibitor

Examples of NO-synthase inhibitors that are suitable for use in the present invention especially comprise a plant extract of the species Vitis vinifera which is sold especially by the company Euromed under the name Leucocyanidines de raisins extra, or by the company Indena under the name Leucoselect®, or finally by the company Hansen under the name Extrait de marc de raisin; a plant extract of the species Olea europaea which is preferably obtained from olive tree leaves and is sold especially by the company vinyals in the form of a dry extract, or by the company Biologia & Technologia under the trade name Eurol BT; and a plant extract of the species Gingko biloba which is preferably a dry aqueous extract of this plant sold by the company Beaufour under the trade name Gingko biloba extrait standard.

6. Agent for Stimulating the Synthesis of Dermal or Epidermal Macromolecules and/or for Preventing Their Degradation

Among the active agents for stimulating dermal macromolecules or for preventing their degradation, mention may be made of those that act:

either on collagen synthesis, such as extracts of Centella asiatica; asiaticosides and derivatives; ascorbic acid or vitamin C and its derivatives; synthetic peptides such as lamin, biopeptide CL or the palmitoyloligopeptide sold by the company Sederma; peptides extracted from plants, such as the soybean hydrolysate sold by the company Coletica under the trade name Phytokine®; and plant hormones such as auxins;

or on elastin synthesis, such as the extract of Saccharomyces cerivisiae sold by the company LSN under the trade name Cytovitin®; and the extract of the alga Macrocystis pyrifera sold by the company SECMA under the trade name Kelpadelie®;

or on glycosaminoglycan synthesis, such as the product of fermentation of milk with Lactobacillus vulgaris, sold by the company Brooks under the trade name Biomin yogourth®; the extract of the brown alga Padina pavonica sold by the company Alban Müller under the trade name HSP3®; and the extract of Saccharomyces cerevisiae available especially from the company Silab under the trade name Firmalift® or from the company LSN under the trade name Cytovitin®;

or on fibronectin synthesis, such as the extract of the zooplankton Salina sold by the company Seporga under the trade name GP4G®;

the yeast extract available especially from the company Alban Müller under the trade name Drieline®; and the palmitoyl pentapeptide sold by the company Sederma under the trade name Matrixil®;

or on metalloprotease (MMP) inhibition, such as, more particularly, MMP 1, 2, 3 or 9. Mention may be made of: retinoids and derivatives, oligopeptides and lipopeptides, lipoamino acids, the malt extract sold by the company Coletica under the trade name Collalift®; extracts of blueberry or of rosemary; lycopene; isoflavones, their derivatives or plant extracts containing them, in particular extracts of soybean (sold, for example, by the company Ichimaru Pharcos under the trade name Flavosterone SB®), of red clover, of flax, of kakkon, or of sage;

or on the inhibition of serine proteases such as leukocyte elastase or cathepsin G. Mention may be made of: the peptide extract of Leguminosa seeds (Pisum sativum) sold by the company LSN under the trade name Parelastyl®; heparinoids; and pseudodipeptides.

Among the active agents that stimulate epidermal macromolecules, such as fillagrin and keratins, mention may be made especially of the extract of lupin sold by the company Silab under the trade name Structurine®; the extract of beech Fagus sylvatica buds sold by the company Gattefosse under the trade name Gatuline®; and the extract of the zooplankton Salina sold by the company Seporga under the trade name GP4G®.

7. Agent for Stimulating the Proliferation of Fibroblasts or Keratinocytes and/or Keratinocyte Differentiation

The agents for stimulating the proliferation of fibroblasts that may be used in the composition according to the invention may be chosen, for example, from plant proteins or polypeptides, extracts, especially of soybean (for example an extract of soybean sold by the company LSN under the name Eleseryl SH-VEG 8® or sold by the company Silab under the trade name Raffermine®); and plant hormones such as giberrellins and cytokinins.

The agents for stimulating keratinocyte proliferation that may be used in the composition according to the invention especially comprise retinoids such as retinol and its esters, including retinyl palmitate; phloroglucinol; extracts of nut cakes sold by the company Gattefosse; and extracts of Solanum tuberosum sold by the company Sederma.

The agents for stimulating keratinocyte differentiation comprise, for example, minerals such as calcium; the extract of lupin sold by the company Silab under the trade name Photopreventine®; sodium beta-sitosteryl sulphate sold by the company Seporga under the trade name Phytocohesine®; and the extract of corn sold by the company Solabia under the trade name Phytovityl®.

8. Dermo-Decontracting Agent

Besides the compound of formula (I) described above, the composition according to the invention may comprise other dermo-decontracting agents, among which mention may be made in particular of alverine and its salts, especially alverine citrate, manganese gluconate, sapogenins such as diosgenin and the natural extracts containing them (such as extracts of wild yam), and also the hexapeptide argireline R sold by the company Lipotec.

9. Tensioning Agent

The term “tensioning agent” means a compound capable of exerting tension on the skin, the effect of which is to temporarily fade out irregularities on the skin's surface, such as wrinkles and fine lines.

Among the tensioning agents that may be used in the composition according to the present invention, mention may be made especially of:

(1) synthetic polymers, such as polyurethane latices or acrylic-silicone latices, in particular those described in patent application EP-1 038 519, such as a propylthio(polymethyl acrylate), propylthio(polymethyl methacrylate) and propylthio(polymethacrylic acid) grafted polydimethylsiloxane, or alternatively a propylthio(polyisobutyl methacrylate) and propylthio(polymethacrylic acid) grafted polydimethylsiloxane. Such grafted silicone polymers are sold especially by the company 3M under the trade names VS 80, VS 70 or LO21.

(2) polymers of natural origin, especially (a) polyholosides, for example (i) in the form of starch derived especially from rice, corn, potato, cassaya, pea, Triticum aestivum wheat, oat, etc. or (ii) in the form of carrageenans, alginates, agars, gelans, cellulose-based polymers and pectins, advantageously as an aqueous dispersion of gel microparticles, and (b) latices consisting of shellac resin, sandarac gum, dammar resins, elemi gums, copal resins and cellulose-based derivatives, and mixtures thereof,

(3) plant proteins and protein hydrolysates, in particular from corn, rye, Triticum aestivum wheat, buckwheat, sesame, spelt, pea, bean, lentil, soybean and lupin,

(3) mixed silicates, especially phyllosilicates and in particular Laponites,

(4) wax microparticles chosen, for example, from carnauba wax, candelilla wax and alfalfa wax,

(5) colloidal particles of mineral filler with a number-average diameter of between 0.1 and 100 nm and preferably between 3 and 30 nm, chosen, for example, from: silica, cerium oxide, zirconium oxide, alumina, calcium carbonate, barium sulphate, calcium sulphate, zinc oxide and titanium dioxide.

10. Anti-Pollution Agent or Free-Radical Scavenger

The term “anti-pollution agent” means any compound capable of trapping ozone, monocyclic or polycyclic aromatic compounds such as benzopyrene and/or heavy metals such as cobalt, mercury, cadmium and/or nickel. The term “free-radical scavenger” means any compound capable of trapping free radicals.

As ozone-trapping agents that may be used in the composition according to the invention, mention may be made in particular of vitamin C and its derivatives including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chlorogenic acid; stilbenes, in particular resveratrol; sulphur-containing amino acid derivatives, in particular S-carboxymethylcysteine; ergothioneine; N-acetyl-cysteine; chelating agents for instance N,N′-bis(3,4,5-trimethoxybenzyl)ethylenediamine or one of its salts, metal complexes or esters; carotenoids such as crocetin; and various starting materials, for instance the mixture of arginine, histidine ribonucleate, mannitol, adenosine triphosphate, pyridoxine, phenyl-alanine, tyrosine and hydrolysed RNA, sold by the Laboratoires Sérobiologiques under the trade name CPP LS 2633-12F®, the water-soluble fraction of corn sold by the company Solabia under the trade name Phytovityl®, the mixture of extract of fumitory and of extract of lemon sold under the name Unicotrozon C-49® by the company Induchem, and the mixture of extracts of ginseng, of apple, of peach, of wheat and of barley, sold by the company Provital under the trade name Pronalen Bioprotect®.

As agents for trapping monocyclic or polycyclic aromatic compounds, which may be used in the composition according to the invention, mention may be made in particular of tannins such as ellagic acid; indole derivatives, in particular 3-indolecarbinol; extracts of tea, in particular of green tea, extracts of water hyacinth or Eichhornia crassipes; and the water-soluble fraction of corn sold by the company Solabia under the trade name Phytovityl®.

Finally, as heavy-metal-trapping agents that may be used in the composition according to the invention, mention may be made in particular of chelating agents such as EDTA, the pentasodium salt of ethylenediaminetetramethylenephosphonic acid, and N,N′-bis(3,4,5-trimethoxybenzyl)ethylenediamine or one of the salts, metal complexes or esters thereof; phytic acid; chitosan derivatives; extracts of tea, in particular of green tea; tannins such as ellagic acid; sulphur-containing amino acids such as cysteine; extracts of water hyacinth (Eichhornia crassipes); and the water-soluble fraction of corn sold by the company Solabia under the trade name Phytovityl®.

The free-radical scavengers that may be used in the composition according to the invention comprise, besides certain anti-pollution agents mentioned above, vitamin E and its derivatives such as tocopheryl acetate; bioflavonoids; coenzyme Q10 or ubiquinone; certain enzymes, for instance catalase, superoxide dismutase, lactoperoxidase, glutathione peroxidase and quinone reductases; glutathione; benzylidenecamphor; benzylcyclanones; substituted napthalenones; pidolates; phytanetriol; gamma-oryzanol; lignans; and melatonin.

11. Agents Acting on the Capillary Circulation

The active agents acting on the capillary circulation (vasoprotective or vasodilating agents) are found especially among flavonoids, ruscogenins, esculosides, escin extracted from common horse chestnut, nicotinates, heperidine methyl chalcone, essential oils of lavender or of rosemary, and extracts of Ammi Visnaga.

12. Agents Acting on the Energy Metabolism of Cells

This expression means active agents acting on: the energy metabolism of the skin, such as, for example, and in a non-limiting manner, ATP synthesis, and also those involved in the respiratory chain of the cell or in the energy reserves. Mention may be made in this respect of coenzyme Q10 (ubiquinone), cytochrome C, creatine or phosphocreatine.

As mentioned previously, the composition according to the invention may also contain UVA and/or UVB screening agents, in the form of organic or mineral compounds, the latter optionally being coated to make them hydrophobic.

The organic screening agents may be chosen especially from: anthranilates, in particular menthyl anthranilate; benzophenones, in particular benzophenone-1, benzophenone-3, benzophenone-5, benzophenone-6, benzophenone-8, benzophenone-9, benzophenone-12 and, preferably, benzophenone-3 (oxybenzone) or benzophenone-4 (Uvinul MS40 available from BASF); benzylidenecamphors, in particular 3-benzylidenecamphor, benzylidenecamphorsulphonic acid, camphor benzalkonium methosulphate, polyacrylamido-methylbenzylidenecamphor, terephthalylidenedicamphor-sulphonic acid and, preferably, 4-methylbenzylidene-camphor (Eusolex 6300 available from Merck); benzimidazoles, in particular benzimidazilate (Neo Heliopan AP available from Haarmann & Reimer), or phenylbenzimidazolesulphonic acid (Eusolex 232 available from Merck); benzotriazoles, in particular drometrizole trisiloxane, or methylenebis-benzotriazolyltetramethylbutylphenol (Tinosorb M available from Ciba); cinnamates, in particular cinoxate, DEA methoxycinnamate, diisopropyl methylcinnamate, glyceryl ethylhexanoate dimethoxycinnamate, isopropyl methoxycinnamate, isoamyl cinnamate and, preferably, ethocrylene (Uvinul N35 available from BASF), octyl methoxycinnamate (Parsol MCX available from Hoffmann La Roche), or octocrylene (Uvinul 539 available from BASF); dibenzoylmethanes, in particular butylmethoxydibenzoylmethane (Parsol 1789); imidazolines, in particular ethylhexyl dimethoxybenzylidene dioxoimidazoline; PABAs, in particular ethyl dihydroxypropyl PABA, ethylhexyldimethyl PABA, glyceryl PABA, PABA, PEG-25 PABA and, preferably, diethylhexylbutamidotriazone (Uvasorb HEB available from 3V Sigma), ethylhexyltriazone (Uvinul T150 available from BASF) or ethyl PABA (benzocaine); salicylates, in particular dipropylene glycol salicylate, ethylhexyl salicylate, homosalate or TEA salicylate; triazines, in particular anisotriazine (Tinosorb S available from Ciba); drometrizole trisiloxane.

The mineral screening agents preferably consist of zinc oxide and/or titanium dioxide, preferably of nanometric size, optionally coated with alumina and/or stearic acid.

The composition according to the invention is advantageously intended to be applied, and preferably is applied, to the areas of the face or forehead that are marked with expression wrinkles and fine lines, and/or on individuals with expression wrinkles and fine lines. Application may be pin point application, if desired, meaning application only to the area to be treated, for example directly to expression wrinkles and/or fine lines to be reduced only. Applicators are known for such purposes.

The wrinkles and fine lines concerned are preferably those lying radially around the mouth and/or the eyes, in particular the crow's-feet wrinkles, and/or lying on the forehead, in particular the “lion” wrinkle, located in the glabella, in between the eyebrows, and/or lying horizontally on the forehead

### EXAMPLES

The invention will now be illustrated with the non-limiting examples that follow. In these examples, the amounts are indicated as percentages by weight unless stated otherwise.

### Example 1

### Preparation of 3-(ethyl(3-hydroxy-3-pentyloctyl)amino)propiophenone

Step 1: Synthesis of 1-(benzylethylamino)octan-3-one

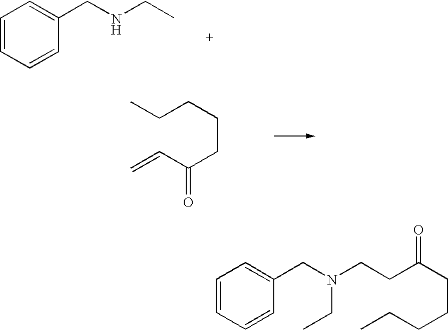

1-Octen-3-one and ethylbenzylamine are reacted in methanol at room temperature. The product obtained is worked up and purified on column of silica. 1.4 g of product (68% yield) are thus recovered. The mass and proton NMR spectrometric analyses are in accordance with the expected product. 300 mg of this intermediate are kept for an activity test. The rest is reacted in the following step.

Step 2: Synthesis of 6-[2-(benzylethylamino)ethyl]-undecan-6-ol

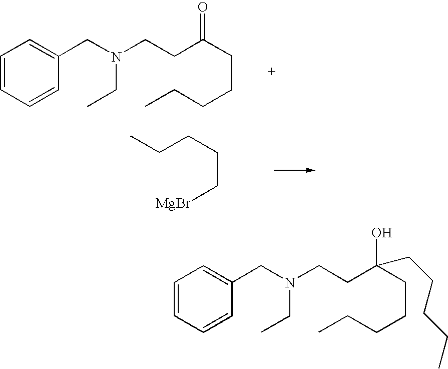

Pentylmagnesium bromide is reacted with the product obtained in step 1, in anhydrous THF, at room temperature. After work-up and purification on silica, 750 mg of product are obtained (50% yield).

The mass and proton NMR spectrometric analyses are in accordance with the expected product.

Step 3: Synthesis of 6-(2-(ethylaminoethyl)undecan-6-ol

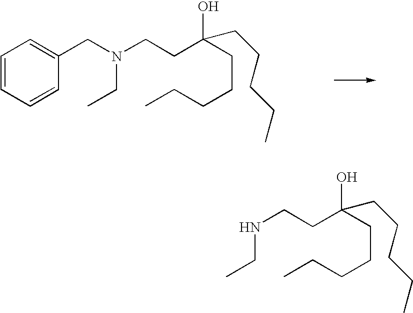

The product obtained in step 2 is debenzylated in ethanol/water under hydrogen pressure in the presence of Pd/C. After filtration through Celite at the end of the reaction, 500 mg of a solid are obtained, which is virtually a single-spot product on thin layer chromatography (100% yield).

The mass and proton NMR spectrometric analyses are in accordance with the expected product.

Step 4: Synthesis of 3-[ethyl(3-hydroxy-3-pentyloctyl)amino]-1-phenylpropan-1-one

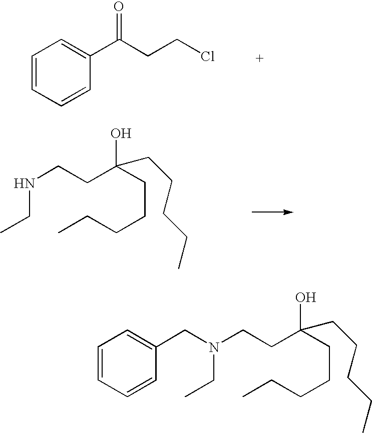

The product obtained in step 3 is reacted with 3-chloropropiophenone in methanol at room temperature for 15 hours. After work-up and purification on silica, 300 mg of product are obtained (78% yield).

The mass spectrometry is in accordance with the expected product.

### Example 2

### Demonstration of the Dermo-Decontracting Effect of the Compounds According to the Invention

The compound of formula (Ia) was tested on a model of dermal equivalent which makes it possible to demonstrate the modulation of the contraction of fibroblasts inserted into a fibrillar collagen gel. This model thus makes it possible to evaluate the relaxing potential of compounds by measuring the delay in contraction of the treated dermal equivalents relative to the control dermal equivalents prepared under the same conditions. Verapamil is used as reference molecule.

1. Preparation of the Dermal Equivalents

Dermal fibroblasts are isolated from human skin explants and cultured in MEM medium+additives (10% foetal calf serum, 1% glutamine, 1% non-essential amino acids, 1% sodium pyruvate, 1% Fungizone and 1% penicillin/streptomycin). The collagen used is obtained from a commercial solution. It is extracted from rat tails and stored in acidic medium at +4° C. It is dialysed beforehand against successive baths of acetic acid of decreasing concentration.

The ingredients below are successively added to a 50 ml centrifuge tube stored in crushed ice:

MEM (1.76×) with or without test compound

foetal calf serum

NaOH (0.1 N)

acetic acid (1/1000)

collagen (3 mg/ml)

1.5×10⁵ fibroblasts/ml of MEM

The mixture is homogenized cautiously, so as to obtain a distribution of the cells in the collagen matrix that is as uniform as possible. It is then distributed in each well of a 12-well culture dish at a rate of 2 ml/well. The final cell concentration is 3×10⁴ cells/dermal equivalent with a final collagen concentration of 1 mg/ml.

The culture dishes are incubated at 37° C./5% CO₂ so as to obtain polymerization and gelation of the collagen. The dermal equivalents thus treated adhere to the dishes and the cells are subjected to mechanical stresses that potentiate their contractile properties. The cells thus cultured in 3D end up under conditions close to those of the dermis. After culturing for three days under these “attached” conditions, the dermal equivalents are detached from their support by gentle agitation. The cells can then exert their tensile forces on the collagen fibres, leading to the expulsion of the interstitial medium and the contraction of the dermal equivalent.

2. Treatment of the Dermal Equivalents

The dermal equivalents are treated with the test compounds (compound Ia and verapamil) from D0, the day of their manufacture, by introducing them into the collagen matrix and into culture medium. The treatment is repeated by changing the culture medium on D3 after releasing the dermal equivalents, which is the starting point of the contraction. Compound (Ia) is applied at a final concentration of 1 μM and verapamil, used as reference molecule, at 10 μM. The control dermal equivalents are placed in contact with the vehicle used for the dilution of compound (Ia) and of verapamil, i.e. DMSO.

The results are the mean of two independent experiments, each experimental point being performed in triplicate.

3. Measurement of the Contraction

The evaluation of the spontaneous contraction of the treated dermal equivalents (treated with compound Ia or verapamil) and control dermal equivalents (without added compound) is performed by measuring their surface area, at different times after the start of the spontaneous contraction.

To do this, a digital image is acquired for each treated or untreated dermal equivalent using a camera (CCD camera—Iris Sony DXC -107P) and on each of them, the small (D1) and large (D2) diameters of the dermal equivalent are measured by computer. The surface area is calculated according to the formula for calculating an ellipse: S=(D1×D2×π)/4. The surface area of one well of a 12-well dish (4 cm²) is considered as the surface area of the dermal equivalent at T0. The percentage of contraction is calculated according to the formula below, in which Si represents the surface area of the dermal equivalent at Ti:

% of contraction=(4−Si)/4×100.

4. Results

The compound of formula (Ia) tested at 1 μm gives rise to a 25% difference in the degree of contraction after 4 days of contact with the fibroblasts, compared with that measured for the control dermal equivalents. For comparative purposes, verapamil tested under the same conditions at 10 μM gives rise to a 32% difference in the degree of contraction.

It was moreover checked that this effect of compound (Ia) did not result from any cytotoxicity.

In addition, alverine tested under the same conditions shows no activity in this test.

This result reflects the dermo-decontracting or dermo-relaxing activity of the compounds according to the invention with respect to normal human fibroblasts, which is comparable with that of verapamil.

Thus, the compounds according to the invention are dermo-decontracting agents that may be used for smoothing out expression wrinkles and fine lines.

### Example 3

### Demonstration of the Calcium-Inhibiting Effect of the Compounds According to the Invention

In order for a substance to be recognized as a calcium-channel inhibitor, it must be able to reduce the intracellular calcium concentration or reduce the binding of calcium to intracellular proteins such as, for example, calmodulin, as is described especially by Galizzi, J. P. et al., J. Biol. Chem. 1987, 262 p. 6947; by Y. Okamiya et al., Eur. J. Pharmacol. 1991, 205, p. 49; by J. A. Wagner et al., J. Neurosci. 1988, 8, p. 3354; by H. R. Lee et al., Life Sci. 1984, 35, p. 721; by Schoemaker H. and Lauger S. Eur. J. Pharmacol. 1985, 111, p. 273 or I. J. Reynolds et al., J. Pharmacol. Exp. Ther. 1986, 237, p. 731.

According to the protocol indicated in these documents, the Assignee determined the IC_{50Ca2+} of inhibition of calcium flux for compounds (Ia), (IIa) to (IId), (IIIa) and (IIIb), in comparison with verapamil. The results are given in the table below. IC_{50Ca2+} represents the 50% inhibitory concentration for the release of Ca²⁺.

IC_{50Ca2+} in μM

| | On cells obtained | |
|---|---|---|
| | from skin fibroblasts | On nerve cells |
| Compounds tested | HDFa | SK-N-SH |
| alverine | ND* | 30 |
| Compound (Ia) | ND | 36 |
| Compound (IIa) | 41 | 29 |
| Compound (IIb) | 31 | 26 |
| Compound (IIc) | 46 | 36 |
| Compound (IId) | 4 | 3 |
| Compound (IIIa) | 30 | 41 |
| Compound (IIIb) | 6 | 14 |

From this table, it is seen that the amines to which the invention applies are indeed calcium-channel inhibitors.

From these tests and from the teaching of patent application EP-1 053 745, it is deduced that the carbonyl amines of formulae (I), (II) and (III) have a high probability of having a beneficial effect on wrinkles and in particular on expression wrinkles.

### Example 4

### Cosmetic Composition

This composition is prepared in a manner that is conventional for those skilled in the art. The amounts indicated are percentages by weight.

| | | |
|---|---|---|
| Compound of formula (Ia) | | 1% |
| Propylene glycol isostearate | | 13% |
| Polyethylene glycol (8 EO) | | 5% |
| Propylene glycol | | 3% |
| Pentylene glycol | | 3% |
| Glyceryl stearate and polyethylene | | |
| glycol stearate (100 EO) | | 5% |
| Oxyethylenated sorbitan monostearate | | 0.5% |
| (20 EO) | | |
| Oxyethylenated (20 EO) | | |
| oxypropylenated (5 PO) cetyl alcohol | | 1% |
| Gelling agents | | 0.5% |
| C₁₂₋₁₅ alkyl benzoates | | 4% |
| Ethanol | | 3% |
| Sodium hydroxide | | 0.12% |
| Preserving agents | | 0.7% |
| Water | qs | 100% |

This fluid is intended to be used in applications once or twice a day to the face and the forehead to attenuate the expression wrinkles and fine lines.

The above written description of the invention provides a manner and process of making and using it such that any person skilled in this art is enabled to make and use the same, this enablement being provided in particular for the subject matter of the appended claims.

All references, patents, applications, tests, standards, documents, publications, brochures, texts, articles, etc. mentioned herein are incorporated herein by reference. Where a numerical limit or range is stated, including carbon, etc. number ranges, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

What is claimed is:

1. A process comprising topically applying to wrinkled skin a composition comprising, in a physiologically acceptable medium, at least one compound of formula (I): 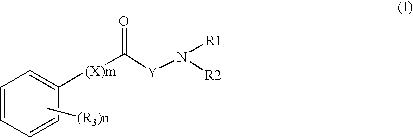
in which:
R₁ denotes a hydrogen atom or a linear or branched, saturated or unsaturated C₁-C₈ alkyl group,
R₂ denotes:
a linear, branched or cyclic, saturated or unsaturated C₁-C₂₀ alkyl group, optionally substituted with an ═O group, and/or with one or more of —CN, —OR, —SR, —NRR′, —COR, —COOR, —CONRR′, —NR—CO—R′, —NR—CO—NR′R″ or —CF₃ groups and/or with one or more halogen atoms and/or with a phenyl group optionally substituted with a C₁-C₄ alkyl group, a C₁-C₄ alkoxy group, a hydroxyl group, a halogen atom and/or a CF₃ group,
an aryl group optionally substituted with at least one of a C₁-C₄ alkyl group, a C₁-C₄ alkoxy group, a hydroxyl group, a halogen atom or a CF₃ group, or
a heterocycle,
in which R, R′ and R″ independently denote a hydrogen atom or a linear or branched C₁-C₄ alkyl group, or an aryl group,
R₃ denotes a linear, branched or cyclic, saturated or unsaturated C₁-C₁₂ alkyl group, or an aryl group, optionally substituted with a hydroxyl group, a C₁-C₄ alkoxy group or a hydrogen atom, a —CN, —OR, —SR, —NRR′, —COR, —COOR, —CONRR′, —NR—CO—R′, —NR—CO—NR′R″ or —CF₃ group or a halogen atom,
in which R, R′ and R″ have the meaning given above,
X is a saturated or unsaturated, optionally substituted linear C₁-C₉ alkylene group,
Y is a saturated or unsaturated, optionally substituted linear C₁-C₁₀ alkylene group,
the substituents of X and Y being chosen independently from —OR, —SR, —NRR′, —COR, —COOR, —CO—NRR′, —NR—CO—R′R″, CF₃ groups and mixtures thereof,
in which R, R′ and R″ have the meaning given above,
m is 0 or 1,
n is 0, 1, 2 or 3,
and the addition salts thereof with an acid.

2. The process according to claim 1, wherein said composition comprises an addition salt of a compound of formula (I) obtained by addition with a mineral acid selected from the group consisting of hydrochloric acid, sulphuric acid, nitric acid and phosphoric acid.

3. The process according to claim 1, wherein said composition comprises an addition salt of a compound of formula (I) obtained by addition with an organic acid elected from the group consisting of succinic acid, fumaric acid, lactic acid, glycolic acid, citric acid and tartaric acid.

4. The process according to claim 1, wherein m=0.

5. The process according to claim 1, wherein n=0.

6. The process according to claim 1, wherein Y is a linear C₁-C₃ alkylene group.

7. The process according to claim 1, wherein R₁ is a C₁-C₃ alkyl group.

8. The process according to claim 1, wherein R₂ is a C₁-C₃ alkyl group substituted with an arylcarbonyl group.

9. The process according to claim 1, wherein said composition comprises the following compound of Formula (Ia):

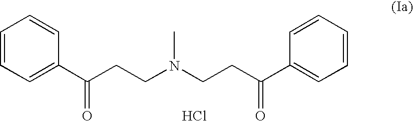

10. The process according to claim 1, wherein the compound of formula (I) represents from 0.1% to 2% of the total weight of the composition.

11. The process according to claim 1, wherein said composition further comprises at least one compound selected from the group consisting of desquamating agents; moisturizers; depigmenting agents; propigmenting agents; antiglycation agents; NO-synthase inhibitors; agents for stimulating the synthesis of dermal or epidermal macromolecules and/or for preventing their degradation; agents for stimulating the proliferation of fibroblasts and/or keratinocytes or for stimulating the differentiation of keratinocytes; dermo-decontracting agents; tensioning agents; antipollution agents and/or free-radical scavengers; agents acting on the capillary circulation; agents acting on the energy metabolism of cells; and mixtures thereof.

12. The process according to claim 1, wherein said composition is applied to the areas of the face or forehead marked with expression wrinkles and fine lines.

13. The process according to claim 1, wehrein said composition is applied to wrinkles and fine lines lying radially around the mouth and/or the eyes and/or horizontally on the forehead and/or lying between the eyebrows.

14. The process according to claim 1, wherein said process smoothes out wrinkles and fine lines.

15. The process according to claim 14, wherein said wrinkles and fine lines are expression wrinkles and fine lines.

16. A compound of formula (II): 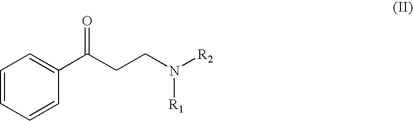
in which:
R₁ is a hydrogen atom or a methyl or ethyl group,
R₂ is a linear or branched C₆-C₂₀ alkyl radical which may be substituted with an oxo group or a hydroxyl group, and the acid addition salts thereof.

17. A compound according to claim 16, having one of the formulae (IIa) to (IIe) below:

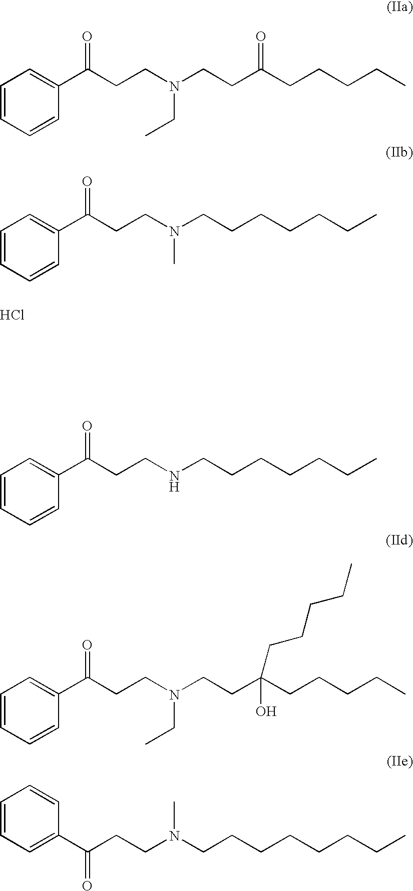

18. A compound corresponding to formula (III): 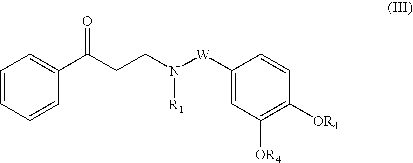
in which:
R₁ denotes a hydrogen atom or a methyl group,
W denotes a linear C₁-C₅ alkylene chain, which may be substituted with an oxo group or a hydroxyl group,
R₄ denotes a hydrogen atom or a methyl group, said compound of formula (III) not comprising an amide function,
and the acid addition salts thereof.

19. A compound according to claim 18, corresponding to one of formulae (IIIa) and (IIIb) below:

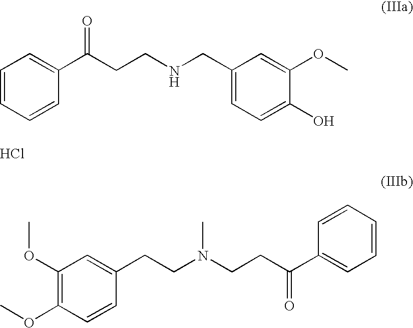

20. A composition comprising, in a physiologically acceptable medium, a compound according to claim 16.

21. A composition comprising, in a physiologically acceptable medium, a compound according to claim 17.

22. A composition comprising, in a physiologically acceptable medium, a compound according to claim 18.

23. A composition comprising, in a physiologically acceptable medium, a compound according to claim 19.
